(19) United States
(12) Patent Application Publication
(10) Pub. No.: US 2022/0307048 A1
(43) Pub. Date: Sep. 29, 2022

(54) ANTIMICROBIAL PEPTIDES

(71) Applicant: Genvor Inc., Dallas, TX (US)

(72) Inventors: Jesse Michael Jaynes, Auburn, AL (US); Clayton C. Yates, Auburn, AL (US)

(21) Appl. No.: 17/700,591

(22) Filed: Mar. 22, 2022

Related U.S. Application Data
(60) Provisional application No. 63/164,832, filed on Mar. 23, 2021.

(51) Int. Cl. C12N 15/82; C07K 14/415

(52) U.S. Cl. CPC C12N 15/8279; C07K 14/415

(57) ABSTRACT

The present invention includes an antimicrobial peptide consisting of, consisting essentially of, or comprising, at least one of SEQ ID NO:1-20, nucleic acids encoding the peptides, methods for using the antimicrobial peptide generating plants which express the peptides sufficient to endow the plants with resistance to pathogenic bacteria and fungi, the use of the nucleic acids to produce peptides by cellular production such as fermentation or plant production systems of the antimicrobial peptide(s), and the use of the isolated antimicrobial peptide(s) in a spray or other mixture to treat plants to protect them from disease.

### CROSS-REFERENCE TO RELATED APPLICATIONS

This application claims priority to U.S. Provisional Application Ser. No. 63/164,832, filed Mar. 23, 2021, the entire contents of which are incorporated herein by reference.

### TECHNICAL FIELD OF THE INVENTION

The present invention relates in general to the field of antimicrobial peptides, and more particularly, to antimicrobial peptides with a high biological activity against a broad range of plant pathogenic bacteria and fungi with reduced toxicity towards non-target species.

### STATEMENT OF FEDERALLY FUNDED RESEARCH

Not applicable.

### INCORPORATION-BY-REFERENCE OF MATERIALS FILED ON COMPACT DISC

The present application includes a Sequence Listing which has been submitted in ASCII format via EFS-Web and is hereby incorporated by reference in its entirety. The ASCII copy, created on Mar. 9, 2022, is named GENV1002 ST25.txt and is 5,783 bytes in size.

### BACKGROUND OF THE INVENTION

Without limiting the scope of the invention, its background is described in connection with antimicrobial peptides.

The welfare of humanity is inextricably bound up with the efficient cultivation of plants. Plant disease is a disruptive and, at times, catastrophic occurrence. For instance, late blight, a disease resulting from an infection by a fungal pathogen, caused the starvation of one million people and forced the immigration of another two million to North America owing to its decimation of the potato crop (the infamous Irish Potato Famine of 1845-60). Clearly, the economic and social impact of plant disease can be substantial. It is estimated that as much as one-third of the total crops lost to humanity can be directly attributed to plant disease.

### SUMMARY OF THE INVENTION

In one embodiment, the present invention includes an antimicrobial peptide consisting of, consisting essentially of, or comprising, at least one of SEQ ID NO:1-20. In one aspect, the antimicrobial peptide is fused to a peptide or polypeptide. In another aspect, the further peptide is a tag, a signal peptide or an antigenic determinant. In another aspect, the antimicrobial peptide is fused to a peptide or polypeptide via a linker.

In another embodiment, the present invention includes a nucleic acid molecule encodes one or more peptides consisting of, consisting essentially of, or comprising, at least one of SEQ ID NO:1-20.

In another embodiment, the present invention includes an expression vector comprising the nucleic acid molecule that encodes a peptide consisting of, consisting essentially of, or comprising, at least one of SEQ ID NO:1-20.

In another embodiment, the present invention includes a host cell which may be grown in cell culture comprising the vector comprising the nucleic acid molecule that encodes a peptide consisting of, consisting essentially of, or comprising, at least one of SEQ ID NO:1-20.

In another embodiment, the present invention includes a method of producing an antimicrobial peptide consisting of, consisting essentially of, or comprising, at least one of SEQ ID NO:1-20 comprising culturing the host cell and collecting the peptide produced. In another aspect, the host cell is a bacteria, fungi, plant, or insect cell.

In another embodiment, the present invention includes a method of treating a plant comprising contacting a plant with an amount of a peptide of at least one of SEQ ID NOS:1-20 sufficient to prevent or treat an infectious disease. In another aspect, the infectious diseases are caused by bacterial or fungal infection. In another aspect, the infectious disease is Botrytis sp., Clavibaceter sp., Colletotrichum sp., Pseudomonas sp., Erwinia sp., Xanthomas sp., Rhizoctonia sp., Verticillium sp., Didymella sp., or Fusarium sp. In another aspect, the plant treated is selected from maize, soybean, sunflower, sorghum, canola, wheat, alfalfa, cotton, rice, barley, or millet. In another aspect, the plant treated by spraying on the plant, leaves, roots, shoots, or soil.

In another embodiment, the present invention includes a kit comprising an antimicrobial peptide consisting of, consisting essentially of, or comprising, at least one of SEQ ID NO:1-20.

In another embodiment, the present invention includes a recombinant expression cassette comprising a nucleic acid molecule having the polynucleotide sequence of a nucleic acid molecule that encodes a peptide consisting of, consisting essentially of, or comprising, at least one of SEQ ID NO:1-20 operably linked to a promoter.

In another embodiment, the present invention includes a host cell comprising the recombinant expression cassette comprising a nucleic acid molecule having the polynucleotide sequence of a nucleic acid molecule that encodes a peptide consisting of, consisting essentially of, or comprising, at least one of SEQ ID NO:1-20 operably linked to a promoter.

In another embodiment, the present invention includes a transgenic plant cell comprising the recombinant expression cassette comprising a nucleic acid molecule having the polynucleotide sequence of a nucleic acid molecule that encodes a peptide consisting of, consisting essentially of, or comprising, at least one of SEQ ID NO:1-20 operably linked to a promoter.

In another embodiment, the present invention includes a transgenic plant comprising the recombinant expression cassette comprising a nucleic acid molecule having the polynucleotide sequence of a nucleic acid molecule that encodes a peptide consisting of, consisting essentially of, or comprising, at least one of SEQ ID NO:1-20 operably linked to a promoter. In another aspect the transgenic plant is maize, soybean, sunflower, sorghum, canola, wheat, alfalfa, cotton, rice, barley, or millet.

In another embodiment, the present invention includes a transgenic seed from the transgenic plant, wherein the transgenic plant is maize, soybean, sunflower, sorghum, canola, wheat, alfalfa, cotton, rice, barley, or millet.

### BRIEF DESCRIPTION OF THE DRAWINGS

For a more complete understanding of the features and advantages of the present invention, reference is now made to the detailed description of the invention along with the accompanying figures and in which:

FIG. 1 shows the activity of peptides of the present invention on different microbial species.

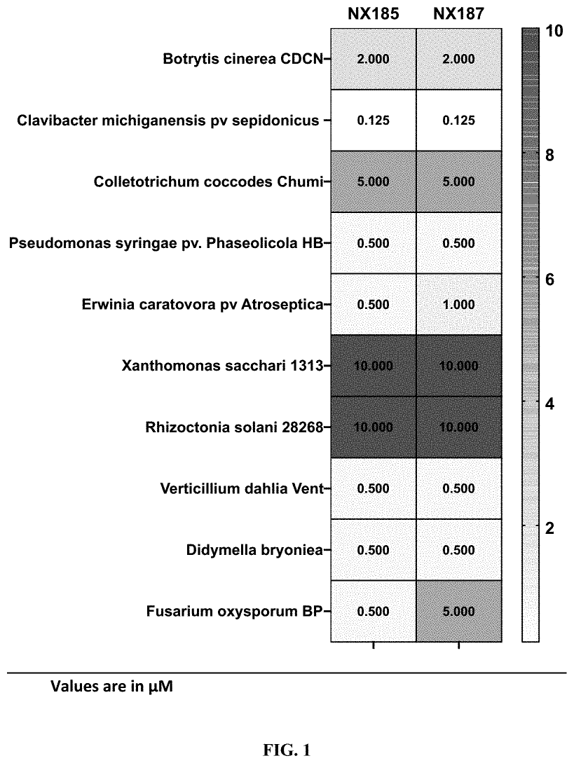

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

These peptides have been experimentally confirmed to exert the desired effect in the course of the present invention and thus provide a broad exemplary basis of the claimed invention. In one embodiment, the peptide of the invention is fused to a further peptide or a polypeptide.

By fusing the peptide of the present invention to a further peptide or polypeptide, a fusion peptide or polypeptide is formed, i.e., an at least bipartite molecule comprising the peptide of the invention. The fusion peptide or polypeptide may exceed the length for a peptide as defined above, i.e., form an amino acid sequence of more than 30 amino acids which is defined as a polypeptide in accordance with the present invention which term is interchangeably used with the term “protein”. Preferably, the further peptide does not have antimicrobial or antiviral activity. Alternatively, the further peptide displays antimicrobial or antiviral activity. Thus, it is conceivable in accordance with the present invention that two inventive peptides form the fusion peptide or fusion polypeptide. The fusion peptide or polypeptide of the present invention can be produced and isolated according to the methods described herein for the production of the peptide of the invention.

In one embodiment, the further peptide is a tag, a signal peptide, an antigenic determinant or a therapeutically active peptide such as a cytokine. Suitable polypeptides which can be fused to the peptide of the invention are polypeptides which may e.g., increase the solubility and/or facilitate the purification of the peptide of the invention. The tag could serve for purification purposes if the peptide is produced by recombinant methods. Exemplary tags in this regard are a 6xHis-tag, an HA-tag or a FLAG-tag which as such are known in the art. On the other hand, the tag could also be used to target the peptide of the invention to an organ or tissue wherein the cells express certain antigens to which the tag binds. Thus, the tag could be, e.g., a peptide ligand for a receptor. Antigenic determinants allow for the purification of the fusion peptides via antibody affinity columns. Signal peptides are short amino acid sequences capable of directing the peptide or protein to which they are attached to different cellular compartments or to the extracellular space.

In another embodiment, the peptide of the invention is fused to the further peptide or polypeptide via a linker. A linker in connection with the present invention is used to connect the peptide of the invention with other peptides or with polypeptides. The linker serves to physically separate, the peptide of the invention and the other peptide or polypeptide and to ensure that neither the peptide of the invention nor the other peptide(s) or polypeptide(s) are limited in their function due to the close vicinity to each other. Depending on the other peptide or polypeptide, the linker can be a peptide bond, an amino acid, a peptide of appropriate length, or a different molecule providing the desired features. The skilled person knows how to design appropriate linker molecules, in particular linker peptides based on his/her common knowledge. For example, peptide linkers can be chosen from the LIP (Loops in Proteins) database (Michalsky et al., 2003). A linker may be appended to the N- or the C-terminus or, if deemed suitable, also to an amino acid apart from the terminal amino acids of the peptide of the present invention. The linker is preferably located at the N-terminus. In one embodiment, the linker is a lysine, glycine, serine, an ether, ester or a disulfide.

In another embodiment, the present invention relates to a nucleic acid molecule encoding the peptide or the fused peptide (fused to another peptide or a polypeptide) of the invention.

As sued herein, the term “nucleic acid molecule”, or “polynucleotide refer to DNA, such as cDNA or genomic DNA, and RNA. Further included are nucleic acid mimicking molecules known in the art such as synthetic or semi-synthetic derivatives of DNA or RNA and mixed polymers. Such nucleic acid mimicking molecules or nucleic acid derivatives according to the invention include phosphorothioate nucleic acid, phosphoramidate nucleic acid, 2′-O-methoxyethyl ribonucleic acid, morpholino nucleic acid, hexitol nucleic acid (HNA) and locked nucleic acid (LNA) (see Braasch and Corey, Chem Biol 2001, 8: 1). LNA is an RNA derivative in which the ribose ring is constrained by a methylene linkage between the 2′-oxygen and the 4′-carbon. They may contain additional non-natural or derivative nucleotide bases, as will be readily appreciated by those skilled in the art.

It will be readily appreciated by the skilled person that more than one or more nucleic acid(s) may encode the peptide of the present invention due to the degeneracy of the genetic code. Degeneracy results because a triplet base code composed of four bases designates each of the 20 proteinogenic amino acids and a stop codon. The possible 4³ possibilities for bases in triplets gives 64 possible codons, meaning that some degeneracy must exist. As a result, some amino acids are encoded by more than one triplet, i.e., by up to six. The degeneracy mostly arises from alterations in the third position in a triplet. This means that nucleic acid molecules having a different sequence, but still encoding the same polypeptide within the scope of the present invention.

The present invention also includes expression vectors comprising the nucleic acid molecule of the invention that encode the peptides herein. Expression vectors include, e.g., plasmids, cosmids, viruses, bacteriophage or another vector used conventionally, e.g., in molecular biology engineering.

The nucleic acid molecule(s) of the present invention may be inserted into several commercially available vectors. Non-limiting examples include prokaryotic plasmid vectors, such as the pUC-series, pBluescript (Stratagene), the pET-series of expression vectors (Novagen) or pCRTOPO (Invitrogen), lambda gt11, pJOE, the pBBR1-MCS series, pJB861, pBSMuL, pBC2, pUCPKS, pTACT1 and vectors compatible with expression in mammalian cells like pREP (Invitrogen), pCEP4 (Invitrogen), pMC1neo (Stratagene), pXT1 (Stratagene), pSG5 (Stratagene), EBO-pSV2neo, pBPV-1, pdBPVMMTneo, pRSVgpt, pRSVneo, pSV2-dhfr, pIZD35, Okayama-Berg cDNA expression vector pcDV1 (Pharmacia), pRc/CMV, pcDNA1, pcDNA3 (Invitrogene), pSPORT1 (GIBCO BRL), pGEMHE (Promega), pLXIN, pSIR (Clontech), pIRES-EGFP (Clontech), pEAK-10 (Edge Biosystems) pTriEx-Hygro (Novagen) and pC1Neo (Promega). Examples for plasmid vectors suitable for Pichia pastoris comprise, e.g., the plasmids pAO815, pPIC9K and pPIC3.5K (all Invitrogen).

The nucleic acid molecule of the present invention may also be inserted into vectors such that a translational fusion with another nucleic acid molecule is generated. The other nucleic acid molecules may encode a protein which may, e.g., increase the solubility and/or facilitate the purification of the protein encoded by the nucleic acid molecule of the invention. Non-limiting examples include pET32, pET41, pET43. Furthermore, the other nucleic acid molecule may encode a peptide or protein which enables for the compensation of the toxic properties of the antimicrobial peptides of the invention which would otherwise harm or kill the host cell.

The vectors may also contain an additional expressible polynucleotide coding for one or more chaperones to facilitate correct protein folding. Suitable bacterial expression hosts comprise e. g. strains derived from BL21 (such as BL21(DE3), BL21(DE3)PlysS, BL21(DE3)RIL, BL21(DE3)PRARE) or ROSETTA®. For vector modification techniques, see Sambrook and Russel (2001). Generally, vectors can contain one or more origins of replication (ori) and inheritance systems for cloning or expression, one or more markers for selection in the host, e.g., antibiotic resistance, and one or more expression cassettes. Suitable origins of replication include, for example, the Col E1, the SV40 viral and the M 13 origins of replication.

The coding sequences inserted in the vector can be, e.g., synthesized by standard methods, or isolated from natural sources. Ligation of the coding sequences to transcriptional regulatory elements and/or to other amino acid encoding sequences can be carried out using established methods. Transcriptional regulatory elements (parts of an expression cassette) ensuring expression in prokaryotes or eukaryotic cells are well known to those skilled in the art. These elements comprise regulatory sequences ensuring the initiation of the transcription (e. g., translation initiation codon, promoters, enhancers, and/or insulators), internal ribosomal entry sites (IRES) (Owens et al., 2001) and optionally poly-A signals ensuring termination of transcription and stabilization of the transcript. Additional regulatory elements may include transcriptional as well as translational enhancers, and/or naturally-associated or heterologous promoter regions. Preferably, the nucleic acid molecule of the invention is operably linked to such expression control sequences allowing expression in prokaryotes or eukaryotic cells. The vector may further comprise nucleotide sequences encoding signal peptides as further regulatory elements. Such sequences are well known to the person skilled in the art. Furthermore, depending on the expression system used, leader sequences capable of directing the expressed polypeptide to a cellular compartment may be added to the coding sequence of the nucleic acid molecule of the invention. Such leader sequences are well known in the art. The present invention also includes vectors that allow the shuttling of DNA between different hosts, such as bacteria-fungal cells or bacteria-animal cells.

An expression vector according to this invention is capable of directing the replication, and the expression of the nucleic acid molecule of the invention and the peptide, fusion peptide or fusion polypeptide encoded thereby. Suitable expression vectors are described above.

The nucleic acid molecules of the invention as described herein above may be designed for direct introduction or for introduction via liposomes, phage vectors or viral vectors (e.g., adenoviral, retroviral) into the cell. Additionally, baculoviral systems or systems based on Vaccinia Virus or Semliki Forest Virus can be used as vector in eukaryotic expression system for the nucleic acid molecules of the invention. Expression vectors derived from viruses such as retroviruses, vaccinia virus, adeno-associated virus, herpes viruses, or bovine papilloma virus, may be used for delivery of the polynucleotides or vector into targeted cell population. Methods which are well known to those skilled in the art can be used to construct recombinant viral vectors; see, for example, the techniques described in Sambrook, 2001 and Ausubel, 2001.

Suitable prokaryotic host cells comprise, e.g., bacteria of the genera Escherichia, Streptomyces, Salmonella or Bacillus. It is of note that in case prokaryotic host cells are used, the vector of the invention comprises the fusion peptide or fusion polypeptide of the invention if the expressed peptide alone would be toxic to the prokaryotic cells.

The present invention can also be expressed in eukaryotic cells. Suitable eukaryotic host cells are, e.g., yeasts such as Saccharomyces cerevisiae or Pichia pastoris. Insect cells suitable for expression are, e.g., Drosophila S2 or Spodoptera Sf9 cells. In order to be able to express the peptide of the invention in sufficient amounts, often, the fusion peptide or the fusion polypeptide is encoded by the vector of the present invention if the expression of the peptide of the invention alone would be toxic to the host cell. This can easily be determined by the skilled person using routine biotechnological methods such as a test expression.

The present inventors have designed a new class of antimicrobial peptides (Table I) that possess superior properties of high biological activity against a broad range of plant pathogenic bacteria and fungi with reduced toxicity towards not target species.

TABLE 1
Antimicrobial Peptides

| GV# | Sequence | #AA | SEQ ID NO: |
|---|---|---|---|
| GV155 | FKLRAKVKIRLRAKIKL | 17 | 1 |
| GV176 | FKIKARLRVKIKARLKL | 17 | 2 |
| GV178 | FRIRAKVKLRIRAKVRL | 17 | 3 |
| GV179 | FRVKARIRLKVKARIRL | 17 | 4 |
| GV181 | KARLKFCFKGLCIKIKVR | 18 | 5 |
| GV182 | KIKARLCLGKFCIKARLK | 18 | 6 |
| GV183 | KAFKKAFKKFKKACFKGL | 32 | 7 |
| | CAKKFKKFAKKFAK | | |
| GV184 | KFAKKFKKFAKKFCFKGL | 32 | 8 |
| | CAFKKAFKKFKKAF | | |
| GV185 | KWCFRVCYRGICYKKCR | 17 | 9 |
| GV186 | RCKKYCIGRYCVRFCWK | 17 | 10 |
| GV187 | KFCWRVCYRGICFKKCR | 17 | 11 |
| GV188 | RCKKFCIGRYCVRWCFK | 17 | 12 |
| GV189 | RFCFKVCFKGICYKKCK | 17 | 13 |
| GV190 | KCKKYCIGKFCVKFCFR | 17 | 14 |
| GV191 | RFCFKVCFKGICFKKCK | 17 | 15 |
| GV192 | KCKKFCIGKFCVKFCFR | 17 | 16 |
| GV193 | KFCFRVCRRGICFRRCR | 17 | 17 |
| GV194 | KFCFRVCFRRICFRRCR | 17 | 18 |
| GV195 | RGGKLCYCRKRFCVCVGK | 18 | 19 |
| GV196 | KGVCVCFRKRCYCLKGGR | 18 | 20 |

### Methodology. Minimum Bactericidal (Fungicidal) Concentration

MBC value represents the lowest concentration that completely kills the population, resulting in no growth in the MBC plate. Results will be determined following the 24 to 96 hours incubation from the Test panels by visual scoring. To determine the minimum bactericidal concentration (MBC) values, check for turbidity (visually) in the wells of the MBC plate. Additionally, a microtiter plate reader to obtain optical density measurements at 595 nm (OD595). Clear wells (OD595<0.1) are evidence of bactericidal activity following a suitable period of incubation.

### MBC Hand Inoculation Testing

Perform the following procedure as one replicate per lab on each strain. Turnaround time requires 24-72 hours for Minimum Bactericidal Concentration (MBC) results.

Inoculum Preparation: The turbidity standard technique is recommended for direct inoculation of the bacterial strains.

Using a cryogenic stock (at −70° C.), streak out a first sub-culture of the bacterial organisms listed above on organism specific media (OSM) as specified in section 4.0.

Incubate at organism specific temperature for required incubation time as stated in section 4.0 and store the plate wrapped in parafilm at 4±1° C.

From the first sub-culture, streak out a second sub-culture on OSA. Incubate at organism specific temperature for required incubation time. The second sub-culture should be used within 32 hours starting from the time it was first removed from incubation.

### For Bacterial Strains

Choose approximately 4-5 large or 5-10 small, well isolated colonies from a secondary culture on OSA.

Emulsify in 3 mL of sterile distilled deionized water in a sterile glass test tube and mix well. If required, adjust to achieve a turbidity equivalent to a 0.5 McFarland standardized suspension to give a desired inoculum density of 1.5×10⁸ cells per mL. Alternatively, measure the absorbance in a spectrophotometer at a wavelength of 625 nm, with water as a blank. Adjust to achieve an absorbance between 0.08-0.13.

Pipette 30 μL of the standardized suspension into 3 mL of cation adjusted Mueller-Hinton broth in Phosphate buffer and mix well. This mixture should give an inoculum density of 1×106 CFU/mL. Serially dilute and spot plate to confirm cell density and to ascertain whether the inoculum meets CLSI requirements.

### For Filamentous Fungi

Take a section of fungus from the agar plate, using sterile forceps or blade and place in a culture flask containing at least 50 mL OSB. Vortex to break up some of the inoculum piece.

Place on a shaker at 110 RPM+ at the strain specific temperature to ensure good aeration for fast growth for up to 7 Days.

Once an adequate amount of growth is observed move the broth culture to a centrifuge tube and spin at 3000×g for 10 minutes, decant and resuspend in OSB up to ˜⅔ the total volume of the container.

Using a sterilized homogenizer insert the probe into the resuspended culture and homogenize until it forms a uniform suspension.

If required dilute the suspension to achieve the target inoculum concentration in the challenge media.

Purity check is performed through examining the spot plated growth control results onto non-selective medium. After 16-20 hours incubation, if a mixed culture is present on the agar plate, retesting is warranted.

### Inoculation of Antibiotic Plates

Inoculate with 20 μL of the inoculum (from step 6.1.6) into each well except the sterility control wells. Pipette 200 μL of organism challenge media OCM into the negative control wells. This step should be completed within 30 minutes after preparing the inoculum from step 6.1.6.

Cover the plate with the lid.

Incubate the inoculated antimicrobial panels at room temperature 22±2° C. incubator for 4 hours.

### Determination of MBC by Serial Dilution and Spot Plating

Following the specified contact times (2 and 4 hours), 100 μL from each well of the challenge plate is placed in the first row of a 96 well microtiter plate.

180 μL of sterile saline is placed in the remaining rows.

A serial dilution (100-10-7) is prepared by transferring 20 μL down each of the 8 rows.

10 μL is removed from each well and spot plated on a prepared OSA plates listed in section 4.0.

The inoculated plates are incubated at 37±2° C. for 16-24 hours, or at 25±2° C. 72-120 hours and the number of colonies will be counted after the appropriate incubation time.

Data is evaluated as total CFU recovered/well.

### Determination of MBC

After the specified contact time, 20 μL from each well of the challenge plate is removed and placed into the corresponding wells of a fresh 96 well Nunc plate containing 180 μL OSM. This will be incubated at the appropriate conditions (24 or 72 hours; Section 4.0). MBC results are determined by scoring +/− for growth based on turbidity of the well.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

What is claimed is:

1. An antimicrobial peptide consisting of, consisting essentially of, or comprising, at least one of SEQ ID NO:1-20.

2. The antimicrobial peptide of claim 1, fused to a further peptide or polypeptide.

3. The antimicrobial peptide of claim 2, wherein the further peptide is a tag, a signal peptide or an antigenic determinant.

4. The antimicrobial peptide of claim 2, fused to the further peptide or polypeptide via a linker.

5. A nucleic acid molecule encoding one or more peptides of claim 1.

6. An expression vector comprising the nucleic acid molecule of claim 5.

7. A host cell which may be grown in cell culture comprising the vector of claim 6.

8. A method of producing the peptide of claim 1 comprising culturing the host cell of claim 7 and collecting the peptide produced.

9. The method of claim 8, wherein the host cell is a bacteria, fungi, plant, or insect cell.

10. A method of treating a plant comprising contacting a plant with an amount of a peptide of at least one of SEQ ID NOS:1-20 sufficient to prevent or treat an infectious disease.

11. The method of claim 10, wherein the infectious diseases are caused by bacterial or fungal infection.

12. The method of claim 10, wherein the infectious disease is Botrytis sp., Clavibaceter sp., Colletotrichum sp., Pseudomonas sp., Envinia sp., Xanthomas sp., Rhizoctonia sp., Verticillium sp., Didymella sp. or Fusarium sp.

13. The method of claim 10, wherein the plant treated is selected from maize, soybean, sunflower, sorghum, canola, wheat, alfalfa, cotton, rice, barley, or millet.

14. The method of claim 10, wherein the plant treated by spraying on the plant, leaves, roots, shoots, or soil.

15. A kit comprising the peptide or fused peptide of claim 1.

16. A recombinant expression cassette comprising a nucleic acid molecule having the polynucleotide sequence of a nucleic acid of claim 5 operably linked to a promoter.

17. A host cell comprising the recombinant expression cassette of claim 16.

18. A transgenic plant cell comprising the recombinant expression cassette of claim 16.

19. A transgenic plant comprising the recombinant expression cassette of claim 16.

20. The transgenic plant of claim 6, wherein the plant is maize, soybean, sunflower, sorghum, canola, wheat, alfalfa, cotton, rice, barley, or millet.

21. A transgenic seed from the transgenic plant of claim 19.


SEQUENCE LISTING (20 sequences)

SEQ ID NO: 1 - protein, 17 aa, Artificial Sequence
  Synthetic GV155
FKLRAKVKIRLRAKIKL

SEQ ID NO: 2 - protein, 17 aa, Artificial Sequence
  Synthetic GV176
FKIKARLRVKIKARLKL

SEQ ID NO: 3 - protein, 17 aa, Artificial Sequence
  Synthetic GV178
FRIRAKVKLRIRAKVRL

SEQ ID NO: 4 - protein, 17 aa, Artificial Sequence
  Synthetic GV179
FRVKARIRLKVKARIRL

SEQ ID NO: 5 - protein, 18 aa, Artificial Sequence
  Synthetic GV181
KARLKFCFKGLCIKIKVR

SEQ ID NO: 6 - protein, 18 aa, Artificial Sequence
  Synthetic GV182
KIKARLCLGKFCIKARLK

SEQ ID NO: 7 - protein, 32 aa, Artificial Sequence
  Synthetic GV183
KAFKKAFKKFKKACFKGLCAKKFKKFAKKFAK

SEQ ID NO: 8 - protein, 32 aa, Artificial Sequence
  Synthetic GV184
KFAKKFKKFAKKFCFKGLCAFKKAFKKFKKAF

SEQ ID NO: 9 - protein, 17 aa, Artificial Sequence
  Synthetic GV185
KWCFRVCYRGICYKKCR

SEQ ID NO: 10 - protein, 17 aa, Artificial Sequence
  Synthetic GV186
RCKKYCIGRYCVRFCWK

SEQ ID NO: 11 - protein, 17 aa, Artificial Sequence
  Synthetic GV187
KFCWRVCYRGICFKKCR

SEQ ID NO: 12 - protein, 17 aa, Artificial Sequence
  Synthetic GV188
RCKKFCIGRYCVRWCFK

SEQ ID NO: 13 - protein, 17 aa, Artificial Sequence
  Synthetic GV189
RFCFKVCFKGICYKKCK

SEQ ID NO: 14 - protein, 17 aa, Artificial Sequence
  Synthetic GV190
KCKKYCIGKFCVKFCFR

SEQ ID NO: 15 - protein, 17 aa, Artificial Sequence
  Synthetic GV191
RFCFKVCFKGICFKKCK

SEQ ID NO: 16 - protein, 17 aa, Artificial Sequence
  Synthetic GV192
KCKKFCIGKFCVKFCFR

SEQ ID NO: 17 - protein, 17 aa, Artificial Sequence
  Synthetic GV193
KFCFRVCRRGICFRRCR

SEQ ID NO: 18 - protein, 17 aa, Artificial Sequence
  Synthetic GV194
KFCFRVCFRRICFRRCR

SEQ ID NO: 19 - protein, 18 aa, Artificial Sequence
  Synthetic GV195
RGGKLCYCRKRFCVCVGK

SEQ ID NO: 20 - protein, 18 aa, Artificial Sequence
  Synthetic GV196
KGVCVCFRKRCYCLKGGR